(19) United States
(12) Patent Application Publication
(10) Pub. No.: US 2015/0112206 A1
(43) Pub. Date: Apr. 23, 2015

(54) HEAT STIMULUS TRANSMISSION EVALUATION METHOD AND EVALUATION METHOD OF DRUG BENEFIT BY THERMAL STIMULATION TRANSMISSION

(71) Applicant: Kenji Ryotokuji, Chiba-shi (JP)

(72) Inventor: Kenji Ryotokuji, Chiba-shi (JP)

(21) Appl. No.: 14/379,850

(22) PCT Filed: Oct. 1, 2012
(86) PCT No.: PCT/JP2012/076056

§ 371 Date: Dec. 29, 2014

(30) Foreign Application Priority Data
Feb. 24, 2012 (JP) 2012-038386

(51) Int. Cl. A61B 5/00; A61B 5/026

(52) U.S. Cl. CPC A61B 5/4848; A61B 5/0261; A61B 5/4884
USPC 600/479

(57) ABSTRACT

According to the present invention, the application of thermal stimulus which are effective on medical conditions are provided in a database. And the increase rate of blood flow before and after applying stimulus to a specific site is compared with the increase rate in the database storing the rate of increase which is previously effective on a medical condition. And when the increase rate of blood flow by application of the thermal stimulus at a selected specific site reaches the increasing blood rate flow which is previously effective on a medical condition, the effect of thermal stimulus is recognized. And the increase rate of blood flow which is effective on a medical condition is 30% or more, preferably 100% or more.

### TECHNICAL FIELD

The present invention relates to a thermal stimulus transmission evaluation method and a method for evaluating drug benefits by thermal stimulus transmission for evaluating improvements in psychological stress, etc., by supplying thermal stimulus to a specific site with an electrical thermal stimulus apparatus.

### BACKGROUND ART

As prior art, Patent Literature 1 is known. Patent Literature 1 states that a thermal stimulus may be easily, accurately as well as objectively evaluated by measuring impedance locus of the skin of an acupuncture patient during acupuncture and inspecting the variation thereof with respect to time. However, there is no description on how the thermal stimulus on medical conditions may be objectively evaluated.

### CITATION LIST

PTL 1 Japanese Unexamined Patent Application Publication No. 2001-112843

### SUMMARY OF INVENTION

### Technical Problem

An object of the present invention is to provide a thermal stimulus evaluation method in which the thermal stimulus on medical conditions can be objectively evaluated. According to the present invention, the increase rate of thermal stimulus which are effective on medical conditions are provided in a database. The increase rate of in blood flow which is previously effective on a medical condition accumulated in the database is compared with the increase rate of in blood flow measured before and after the application of thermal stimulus to a specific site. The drug supplied in a body may be evaluated by using the increase rate in blood flow.

### Solution to Problem

In a thermal stimulus evaluation method to evaluate the thermal stimulus applied to a specific site on a skin surface,

the thermal stimulus evaluation method comprises;
step for measuring a blood flow with a laser Doppler tissue blood flowmeter mounted at the center of an inside wrist joint, before and after the application of thermal stimulus to a specific site that is selected for the application of thermal stimulus;
step for comparing the increase rate of blood flow before and after the application of thermal stimulus with the increase rate in the database storing the increase rate of blood flow which is previously effective on a medical condition,
when the increase rate of blood flow by application of the thermal stimulus at a selected specific site reaches the increase rate in blood flow which is previously effective on a medical condition, the effect of thermal stimulus is recognized, wherein the increase rate of blood flow which is previously effective on a medical condition is 30% or more, preferably 100% or more.

In a thermal stimulus evaluation method to evaluate the thermal stimulus applied to a specific site on a skin surface,

the thermal stimulus evaluation method comprises;
step for measuring a blood flow with a laser Doppler tissue blood flowmeter mounted at the center of an inside wrist joint, before and after the application of thermal stimulus to a specific site that is selected for the application of thermal stimulus;
step for comparing the increase rate of blood flow before and after the application of thermal stimulus with the increase rate in the database storing the increase rate of blood flow which is previously effective on a medical condition; and
when the increase rate of blood flow by application of the thermal stimulus at a selected specific site not reaches the increasing rate of blood flow which is previously effective on a medical condition, the effect of thermal stimulus is not recognized, in this case.
the application of the thermal stimulus is carried out by varying at least one of a thermal stimulus temperature, thermal stimulus time period, a thermal stimulus application method, a pattern of thermal stimulus and a thermal stimulus method,
wherein the increase rate of blood flow which is previously effective on a medical condition is 30% or more, preferably 100% or more.

In the thermal stimulus evaluation method, wherein the drug is supplied into a body, and at the time of supplying of the drug, the increase rate of blood flow by application of the thermal stimulus at a selected specific site is preferred to reach the increase rate in blood flow which is previously effective on a medical condition.

In the thermal stimulus evaluation method, the specific site is at least one site of (1) a site between the first and the second metatarsal heads of right and left soles, (2) a site between the second and the third metatarsal heads and (3) a site where an extended line of inner periphery between the first and second and a perpendicular line of an inner ankle crosses.

### Advantageous Effects of Invention

According to the present invention, the increase rate of thermal stimulus which are effective on medical conditions are provided in a database. And the increase rate of blood flow before and after applying stimulus to a specific site is compared with the increase rate in the database storing the rate of increase which is previously effective on a medical condition. And when the increase rate of blood flow by application of the thermal stimulus at a selected specific site reaches nearly the same as or higher than the increase rate in blood flow the increase rate in blood rate flow which is previously effective on a medical condition, the effect of thermal stimulus is recognized. Wherein, the increase rate of blood flow which is effective on a medical condition is 30% or more, preferably 100% or more. And, after the increase rate of blood flow by application of the thermal stimulus at a selected specific site reaches is nearly the same as or higher than the increase rate in blood flow which is previously effective on a medical condition, the drug is supplied into a body.

### BRIEF DESCRIPTION OF THE DRAWINGS

FIG. 1 is a schematic view of a thermal stimulus applied site at sole.

FIG. 2 is a database showing a change rate of blood flow on a specific site.

FIG. 3 is a database showing a change rate of blood flow on medical conditions;

FIG. 4 is a database showing a rate of increase in 11 patients.

FIG. 5 a schematic view of an electrical thermal stimulus apparatus according to the invention.

FIG. 6 is a schematic view of a circuit diagram of the electrical thermal stimulus apparatus.

FIG. 7 is a schematic view of a arrangement of function in the electrical thermal stimulus apparatus according to the invention.

FIG. 8 shows a thermal stimulus waveform and cycle of a thermal stimulus waveform which is obtained by controlling a thermal stimulus apparatus.

FIG. 9 is a schematic view of a thermal stimulus protocol to thermal stimulus applied sites of a body surface in foot sole of right and left.

### DESCRIPTION OF EMBODIMENTS

The present invention relates to a thermal stimulus transmission evaluation method and a method for evaluating drug benefits by thermal stimulus transmission for evaluating thermal stimulus applied to a specific site on a skin surface by controlling an electrical thermal stimulus apparatus so as to treat medical conditions such as psychological stress.

A thermal stimulus evaluation method to evaluate the thermal stimulus applied to a specific site on a skin surface is carried by controlling an electrical thermal stimulus controller so as to treat medical conditions such as psychological stress.

The thermal stimulus evaluation method comprises as follows;

A blood flow with a laser Doppler tissue blood flowmeter mounted at the center of an inside wrist joint, before and after the application of thermal stimulus to a specific site that is selected for the application of thermal stimulus is measured.

The increase rate of the blood flow before and after the application of thermal stimulus is obtained. The increase rate of blood flow before and after the application of thermal stimulus step is compared with the increase rate in the database storing the increase rate of blood flow which is previously effective on a medical condition, And when the increase rate of blood flow by application of the thermal stimulus at a selected specific site reaches is nearly the same as or higher than the increase rate blood flow which is previously effective on a medical condition, the effect of thermal stimulus is recognized, and the increase rate of blood flow is effective on a medical condition is 30% or more, preferably 100% or more. And, after the increase rate of blood flow by application of the thermal stimulus at a selected specific site reaches is nearly the same as or higher than the increase rate in blood flow which is previously effective on a medical condition, the drug is supplied into a body.

The thermal stimulus transmission evaluation method includes the following steps.

(1) Step of Examining Medical Conditions such as Psychological Stress

Examination categories of examining medical conditions such as psychological stress include blood pressure, a deep body temperature, salivary amylase and a heart rater (arterial age) immediately before stimulus application after a subject is kept quiet in a face-up position for twenty minutes in consideration of the effect on psychological stress through an autonomic nerve.

(2) Step of selecting at a specific site for application of the thermal stimulus.

The specific site comprises at least one part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

(3) A blood flow is measured with a laser Doppler tissue blood flowmeter mounted at the center of an inside wrist joint, before and after the application of thermal stimulus to a specific site. And the rate of increase of blood flow applying stimulus to a specific site is obtained before and after the application of thermal stimulus. And the increase rate before and after the application of stimulus is compared with the increase rate in the database storing the rate of increase which is previously effective on a medical condition.

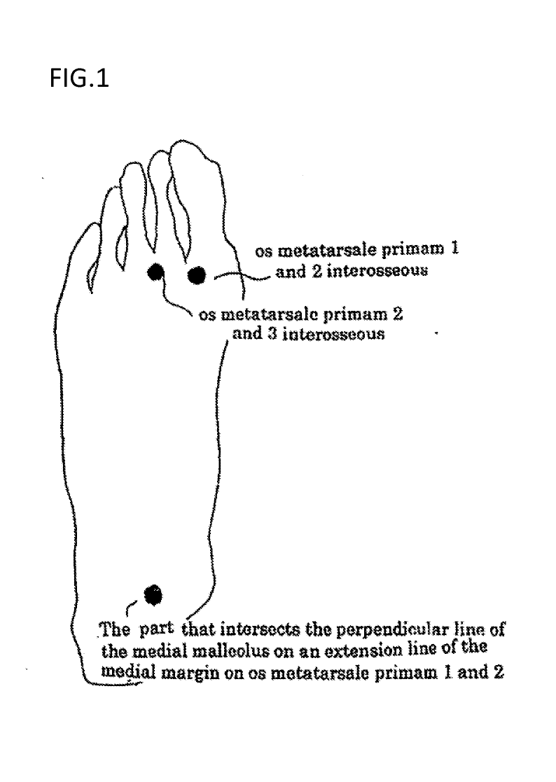

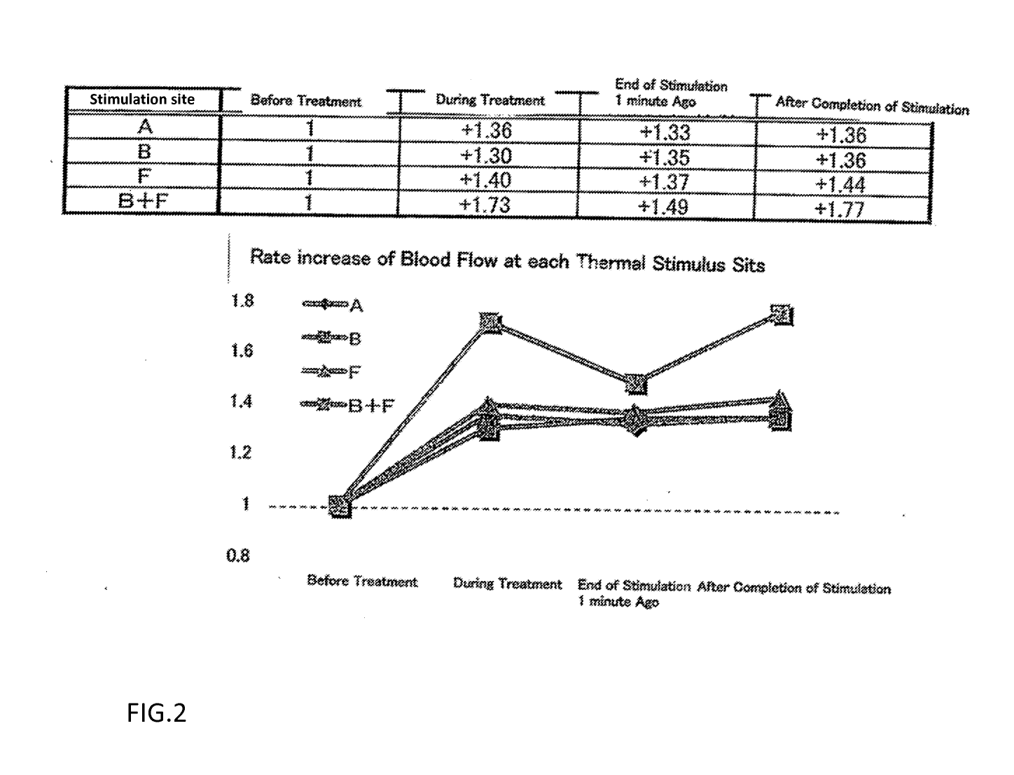

FIG. 2-4 shows the database the rate of increase of the blood flow which is previously effective on a medical condition. FIG. 2 shows the rate increase of the blood flow in the thermal stimulus sites in FIG. 1. In the table, (A) is os metatarsale primam 1 and 2 interosseous, (B) is part os metatarsale primam 2 and 3 interosseous, and (F) is part that intersects the perpendicular line of the medial malleolus on an extension line of the medial margin on os metatarsale primam 1 and 2. The favorable combination is (A) and (F). The rate increase in A is +1.30 or more, the rate increase in B is +1.30 or more, the rate increase in F is +1.30 or more and the rate increase in A+F is +1.40 or more.

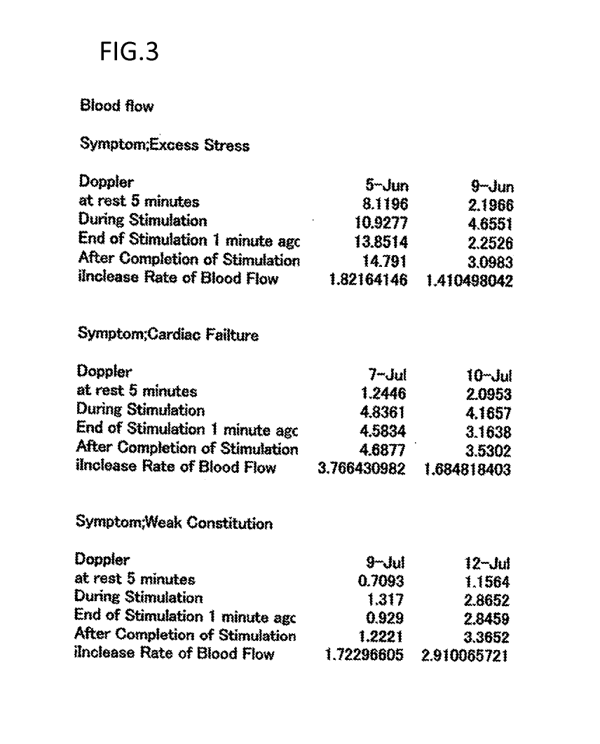

FIG. 3 is a database indicating the increase rate in blood flow in medical conditions. As for the examples of medical conditions, the increase rate in blood flow in excess stress, cardiac failure and weak constitutions are shown. It is found that the increase rate in blood flow for excess stress is +1.4 or more, the increase rate in blood flow for cardiac failure is +1.6 or more, and the increase rate in blood flow for weak constitutions is +1.7 or more.

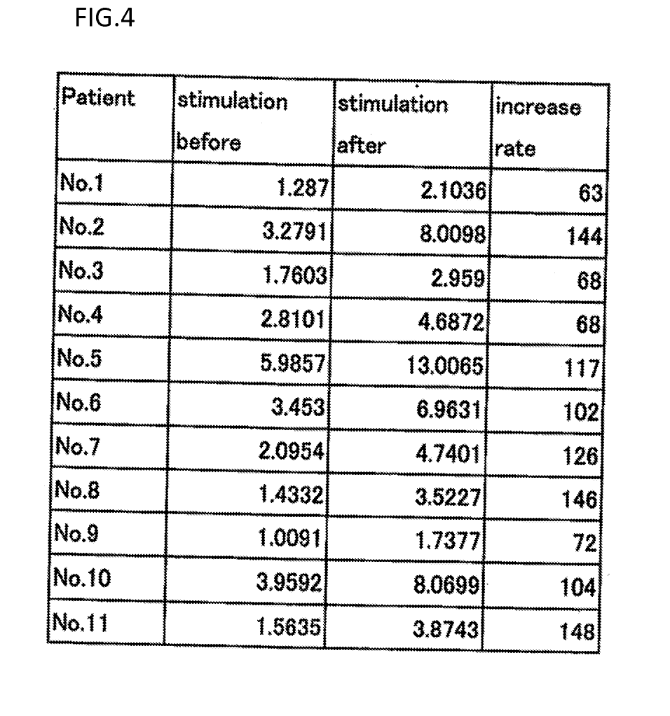

FIG. 4 is a database indicating the increase rate in blood flow of eleven patients. The effects were recognized with the increase rate in blood flow of +1.6 or more.

The increase rate in blood flow at the measurement before and after the application of thermal stimulus to a specific site are compared with the increase rate in blood flow in a database, which is provided with the increase rate in blood flow recognized as effective on medical conditions. And the thermal stimulus is evaluated based on the increase rate in blood flow.

It is desirable to measure the volume of blood flow by the laser Doppler tissue rheometer which is attached to an inner and central part of a wrist joint. In a laser tissue blood flowmeter ALF21D (manufactured by ADOVANS), when a living tissue is irradiated with semiconductor laser light (whose wavelength is 780 nm), light reflected from the tissue is converted into an electric signal and the electric signal is processed, thereby obtaining the blood flow information. A C type laser probe (10 mm in diameter, 3 mm in thickness, 2 mm2 in a laser irradiation area, and 1 mm in measurement depth) of the laser tissue blood flowmeter ALF21D was attached to a central part of a wrist joint horizontal line of a healthy adult, and change of the blood flow was measured, taking a 15-minute rest after a stimulus.

(4) An electrical thermal stimulus controller controls the increase rate of blood flow before and after the application of stimulus. It is preferable that an electrical thermal stimulus controller that is built into an electrical thermal stimulator is driven so as to vary at least one of a thermal stimulus temperature, thermal stimulus time period, a thermal stimulus application method, a pattern of thermal stimulus and a condition of thermal stimulus.

(5) The increase rate of blood flow before and after thermal stimulus application is compared with the increase rate in a database storing the increase rate which is previously effective on medical conditions. The thermal stimulus is evaluated based on the increase rate of blood flow.

Based on the difference of the increase rate between the increase rate of blood flow before and after thermal stimulus application and the increase rate which is previously effective on a medical condition, the effect of thermal stimulus by the application of thermal stimulus is determined. When the increase rate of blood flow by the application of stimulus reaches the increase rate which is previously effective on a medical condition, the effect of the application of thermal stimulus is recognized.

When the increase rate by application of thermal stimulus not reaches the increase rate which are previously effective on medical conditions, it is preferable that the electrical thermal stimulus controller is driven so as to vary at least one of a thermal stimulus temperature, thermal stimulus time period, a thermal stimulus application method, a pattern of thermal stimulus and a condition of thermal stimulus.

Again, the increase rate in blood flow by the application of thermal stimulus to the site is compared the increase rate which is previously effective on a medical condition in the database When the increase rate of blood flow by the application of stimulus reaches the increase rate which is previously effective on a medical condition, the effect of the application of thermal stimulus is recognized.

The method for evaluating drug benefits by thermal stimulus transmission comprises as follows;

A laser Doppler tissue blood flowmeter mounted at the center of an inside wrist joint is provided to measure blood flow. A blood flow before and after the application of thermal stimulus to a specific site that is selected for the application of thermal stimulus is measured with a laser Doppler tissue blood flowmeter mounted at the center of an inside wrist joint.

The increase rate in blood flow measured before and after the application of thermal stimulus to a specific site is compared with the increase rate in blood flow in the database storing blood flow which is previously effective on medical conditions.

The drug is supplied in the body. And at the time of supplying of the drug, the increase rate of blood flow by application of the thermal stimulus at a selected specific site is preferred to reach the increase rate in blood flow which is previously effective on a medical condition.

On the other hand, when the increase rate of blood flow before and after application of the thermal stimulus at a selected specific site not reaches the increase rate of blood flow which is previously effective on a medical condition, the effect by the supplying the drug is not recognized. In this case, the thermal stimulus at a selected specific site is applied by changing the thermal stimulus conditions. And when the increase rate of blood flow before and after application of the thermal stimulus at a selected specific site reaches the increase rate of blood flow which is previously effective on a medical condition, the effect of thermal stimulus is recognized, and the effect by the supplying the drug is recognized. Wherein the drug may be supplied by changing the dose amount and/or an alternate drug.

The sites of the thermal stimulus applied a body surface comprises at least two of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

(6) “Tanden” breathing method may be further performed in the thermal stimulus evaluation.

The “Tanden” is located at about one first length down from the navel. In the Tanden breathing method, breathing is performed slowly while bulging out our abdomen. As the diaphragm lowers and the thorax expands further, large breaths can be taken. With this large breathing, attention is paid on inhaling and exhaling breaths with a long interval in-between to empty mind (mindlessness). Accordingly, autonomic nerve which is disturbed by stress can be adjusted.

FIG. 5 a schematic view of an electrical thermal stimulus apparatus according to the present invention.

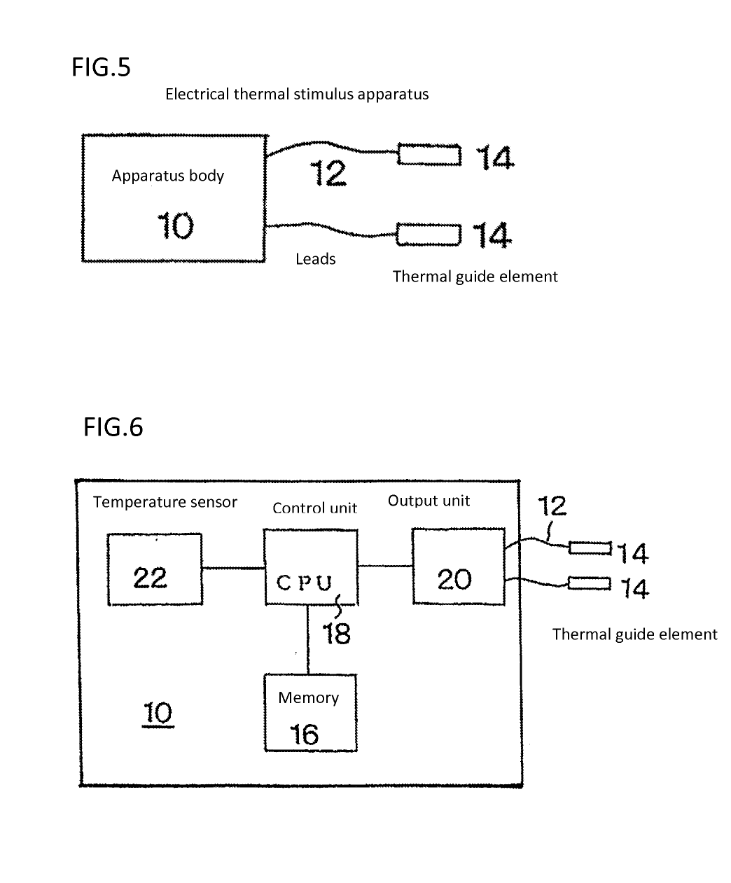

FIG. 6 is a schematic view of a circuit diagram of the electrical thermal stimulus apparatus.

An electrical thermal stimulus apparatus comprises the apparatus boy 10 comprising a control function, and thermal guide element 14 which is connected to the apparatus body 10 by a lead 12. As shown in FIG. 5, the apparatus body 10 comprises a memory unit 16, in which the thermal stimulus patterns are stored, a control unit (CPU) 18, which reads out a thermal stimulus pattern from the memory unit 16, and an output unit 20, which supplies the thermal stimulus pattern to the thermal stimulus guide element 14 for a thermal stimulus. A thermal stimulus is applied to a thermal stimulus applied site, according to the thermal stimulus pattern.

A control unit (CPU) 18 is connected to the memory unit 16. A thermal stimulus pattern for obtaining a stimulus condition equivalent to that obtained from combustion of moxa is stored in the memory unit 16. The control unit (CPU) 18 reads out the thermal stimulus pattern from the memory unit 16, and controls an output to a thermal stimulus guide element(s) based on detection of a emperature sensor 22, and outputs the thermal stimulus pattern to the thermal stimulus guide element(s).

The apparatus body 10 is connected to two or more thermal stimulus guide elements for a thermal stimulus in order to supply the thermal stimulus pattern to at least two different a thermal stimulus applied site. In this manner, the selected thermal stimulus pattern is applied to the thermal stimulus applied site through the thermal guide elements for thermal stimulus.

A temperature sensor 22 is provided in a position(s) which is correlated with a temperature of an affected area near the heating elements. The thermal stimulus guide elements for a thermal stimulus have a structure set forth below. The thermal stimulus guide element 14 for a thermal stimulus comprises a casing of apparatus body, a heater which is provided in the casing, and is used as a source of heating for applying thermal stimulus, a heat conduction board, which is provided on a lower face of the casing and which conducts heat of the heater to a skin of a patient, and a seal board provided on an upper face of the casing.

The thermal stimulus guide elements are provides a heat conduction board made of at least two kinds of materials. In the example, the heat conduction board use aluminum and platinum.

A temperature sensor is provided in a predetermined position of the housing which is in contact with a thermal stimulus applied site of a human body, and detects the temperature of the position, so as to send a detection signal to a sensor amplifier. The control unit (CPU) controls an output of an electric power generating circuit so that the temperature of the portion, which is in contact with a skin surface of a human body contact, may not exceed a predetermined temperature.

When the heating temperature of the thermal stimulus guide elements detected by the temperature sensor is equal to or lower than a reference temperature, a positive side period of a pulse signal is controlled so as to be long and a negative side period of the pulse signal is controlled so as to be short, according to the output of the temperature sensor. On the contrary, when it is in a state at the reference temperature, a positive side period is controlled so as to be short and a negative side period thereof is controlled so as to be long.

The method of operation in the thermal stimulus apparatus is as follows.

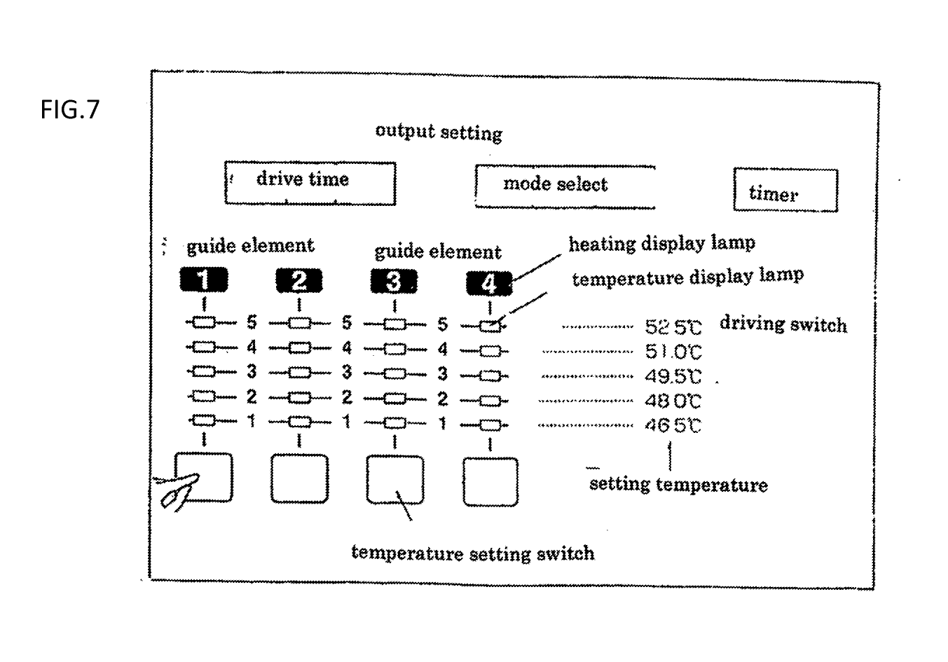

The case where the guide elements are fixed to two thermal stimulus applied sites, which are different from one another, will describe below. The guide elements are fixed to the selected thermal stimulus applied sites, and electric power is supplied from the electric thermal stimulus apparatus. Here, in temperature setting and stimulus application method of the thermal stimulus guide elements, when the number of thermal stimulus applied site is two, the thermal stimulus guide elements 1, and 2 are used (FIG. 7). The temperature is adjusted by a temperature setting switch. Every time the switch is pushed, the temperature of the elements is controlled in a range of from 46.5 to 52.5 in order of 1-2-3-4-5.

A mode selection button for selecting a warming mode, that is, an alternate mode or a sequential mode, is provided therein. In the alternate mode, heating and pausing of the two guide elements 1 and 2 are alternatively repeated. In the sequential mode, thermal stimulus of the two guide elements which are different from one another is carried out one by one in order. In this way, thermal stimulus is non-simultaneously applied to the two different sites independently of one another. The four guide elements is uses for heating (worming) the four sites. In addition, a mode of a heating interval (time) can be chosen. The alternate mode can be chosen. After, a interval mode (short, long) can be chosen. For example, in the alternate mode and short time, the guide elements 1 and 2 is heated alternately, and after five second (at time, after 10 second, also at long time, after 15 second), the heating may be stopped. In the sequential mode, the guide elements 1 and 2 is heated for 7.5 second sequentially.

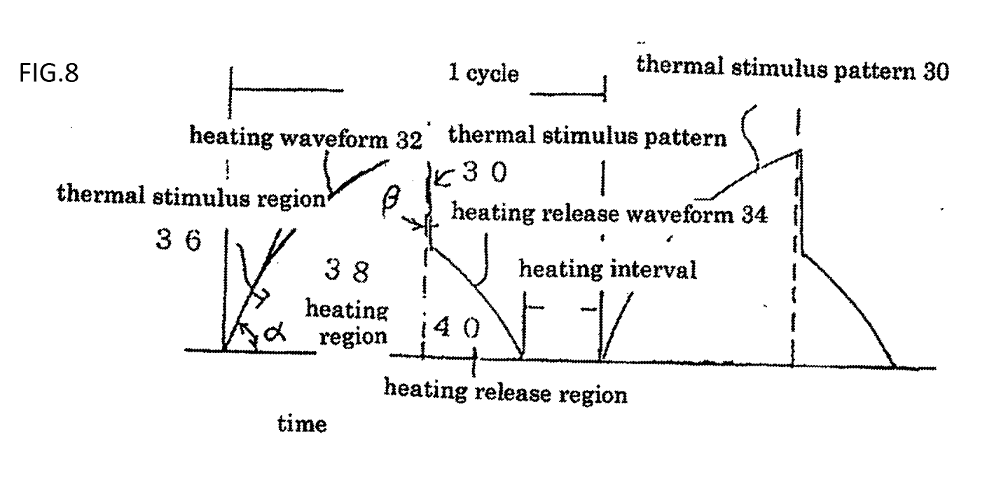

FIG. 8 shows the pattern of the thermal stimulus. The pattern of the thermal stimulus is read out from the controller. The pattern read out of the thermal stimulus provides a thermal waveform 8 and a interval 7. The thermal stimulus waveform includes a heating waveform 32 obtained by heating it to a determined peak temperature, for example, 50±5 degrees Celsius, and a thermal release waveform 34 which is formed by stopping heating after it reaches the peak temperature.

The intensity of the thermal stimulus may be varying the thermal stimulus zone. The intensity of the thermal stimulus is obtained by increasing an area of the warming zone, by increasing a peak temperature, by increasing the gradient of the rising in the heating waveform, by decreasing an area of thermal release zone, by decreasing the gradient of the falling in the release waveform, by decreasing the time of interval, by increasing an time of the thermal stimulus pattern.

The thermal stimulus pattern desirably includes non-simultaneously and independent thermal stimulus waveforms whose phases are shifted so that the patterns of thermal stimulus do not substantially overlap each other at a least different two thermal stimulus applied site. The thermal stimulus is desirably a thermal stimulus.

The first thermal stimulus pattern comprises the thermal stimulus waveform configured by the heating waveform and a thermal release waveform, and an interval to the following thermal stimulus waveform in one cycle of the thermal stimulus patterns,

The second thermal stimulus pattern comprises the thermal stimulus waveform for a time in the interval of first thermal stimulus pattern.

The one cycle of the thermal stimulus pattern is preferably repeated at 10 to 15 minutes. And, after the cycle of the thermal stimulus pattern is repeated at 10 to 15 minutes, and the cycle of the thermal stimulus pattern is repeated at 10 to 15 minutes.

The thermal stimulus pattern and the thermal stimulus waveform are stored in memory.

### EXAMPLE

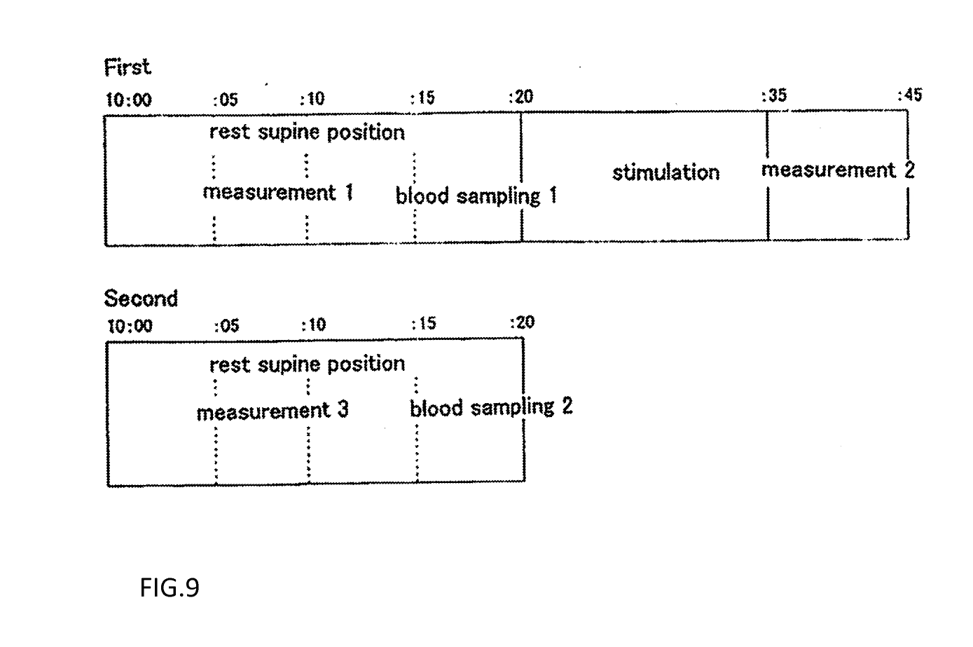

The thermal stimulus apparatus is manufactured by BESTEC., Co Ltd, and the thermal stimulus is carry out by using the thermal stimulus apparatus. And, the protocol of the thermal stimulus is shown in FIG. 9.

The thermal stimulus is carry out applying on thermal guide element at the specific site. The thermal guide element has a diameter; 10 mm and is set at a peak temperature; 50±5 degrees Celsius. The subjects are men and women of adult.

The thermal stimulus applied sites of a body surface preferably comprises at least one of part of os metatarsale primam 1 and 2 interosseous, part os metatarsale primam 2 and 3 interosseous, and part that intersects the perpendicular line of the medial malleolus on an extension line of the medial margin on os metatarsale primam 1 and 2 in foot sole of right and left.

The above-noted rates of increase which are previously effective on medical conditions stored in a database in FIG. 2 to FIG. 4, are recognized as effective on the following medical conditions.

1. Thermal Stimulus to Site so as to Improve Stress (Parasympathetic Nerve is Predominant) Based on Deep Body Temperature, Stress Indicator and Systolic Blood Pressure

A deep body temperature increased at all the sites tested in this experiment. Among them, particularly at a site between the second and the third metatarsal heads, an increase in a temperature by 2.0625° C. is recognized after stimulus. Salivary amylase is one indicator for evaluating stress, and it was shown that salivary amylase was likely to increase after stimulating a sole junction along a perpendicular line between the extended line between the first and second toes, and an inner ankle. Also, it was suggested that cortisol decreased at the same site.

It was recognized that systolic blood pressure decreased significantly by 13 mmHg at a site between the first and second metatarsal heads.

2. Thermal Stimulus to Site so as to Improve Liver Function

It was shown that liver functions were likely to improve by stimulating a sole junction along a perpendicular line between the extended line between the first and second toes, and an inner ankle.

3. Thermal Stimulus on Site so as to Prevent Arteriosclerosis

It was suggested that the blood pressure was decreased further (prevention of arteriosclerosis) by continuously applying thermal stimulus between the first and second metatarsal heads.

4. Thermal Stimulus to Site so as to be Effective on Diabetes

It was confirmed that glucose levels decreased by 5.00 to 5.33 between the first and second metatarsal heads and between the second and third metatarsal heads.

5. Thermal Stimulus to Site so as to Accelerate Peristaltic Motion and Absorption of Intestines

It was found that the secretional capacity of gastrin as a gastrointestinal hormone accelerated significantly when stimulating a sole junction along a perpendicular line between the extended line between the first and second toes, and an inner ankle, which suggested that digestion absorption and the peristaltic motion of intestines would accelerate.

6. Thermal Stimulus to Site so as to Turn “ON” Genes of Longevity

It was shown that Adiponectin was likely to increase after stimulating a site between the first and second metatarsal heads. Adiponectin is considered as one of the substances that activate Sirtuin genes, and is one of the genes related to longevity. Thus, it is suggested that the genes related to longevity are turned ON by stimulating two locations on a sole.

7. Thermal Stimulus to Site for Dieting Effect and Obesity Prevention

Secretional capacities decreased in order of a site between the first and second metatarsal heads and a sole junction along a perpendicular line between the extended line between the first and second toes, and an inner ankle.

### INDUSTRIAL APPLICABILITY

Evaluation of thermal stimulus transmission and evaluation of drug benefits

1-7. (canceled)

8. A thermal stimulus evaluation method to evaluate the thermal stimulus applied to a specific site on a skin surface, the thermal stimulus evaluation method comprising:
a step of measuring a blood flow with a laser Doppler tissue blood flowmeter mounted at the center of an inside wrist joint, before and after the application of thermal stimulus to a specific site that is selected for the application of thermal stimulus; and
a step of comparing the increase rate of blood flow before and after the application of thermal stimulus with the increase rate in the database storing the increase rate of blood flow which is previously effective on a medical condition,
when the increase rate of blood flow by application of the thermal stimulus at a selected specific site reaches the increase rate in blood flow which is previously effective on a medical condition, the effect of thermal stimulus is recognized,
wherein the increase rate of blood flow which is previously effective on a medical condition is 30% or more, preferably 100% or more.

9. A thermal stimulus evaluation method to evaluate the thermal stimulus applied to a specific site on a skin surface, the thermal stimulus evaluation method comprising:
a step for measuring a blood flow with a laser Doppler tissue blood flowmeter mounted at the center of an inside wrist joint, before and after the application of thermal stimulus to a specific site that is selected for the application of thermal stimulus; and
a step of comparing the increase rate of blood flow before and after the application of thermal stimulus with the increase rate in the database storing the increase rate of blood flow which is previously effective on a medical condition; and when the increase rate of blood flow by application of the thermal stimulus at a selected specific site not reaches the increasing rate of blood flow which is previously effective on a medical condition, the effect of thermal stimulus is not recognized, in this case, the application of the thermal stimulus is carried out by varying at least one of a thermal stimulus temperature, thermal stimulus time period, a thermal stimulus application method, a pattern of thermal stimulus and a thermal stimulus method,
wherein the increase rate of blood flow which is previously effective on a medical condition is 30% or more, preferably 100% or more.

10. The thermal stimulus evaluation method according to claim 8, wherein
the drug is supplied into a body, and at the time of supplying of the drug, the increase rate of blood flow by application of the thermal stimulus at a selected specific site is preferred to reach the increase rate in blood flow which is previously effective on a medical condition.

11. The thermal stimulus evaluation method according to claim 8, wherein
when the specific site is at least one site of (1) a site between the first and the second metatarsal heads of right and left soles, (2) a site between the second and the third metatarsal heads and (3) a site where an extended line of inner periphery between the first and second and a perpendicular line of an inner ankle crosses.

12. The thermal stimulus evaluation method according to claim 9, wherein
the drug is supplied into a body, and at the time of supplying of the drug, the increase rate of blood flow by application of the thermal stimulus at a selected specific site is preferred to reach the increase rate in blood flow which is previously effective on a medical condition.

13. The thermal stimulus evaluation method according to claim 9, wherein
when the specific site is at least one site of (1) a site between the first and the second metatarsal heads of right and left soles, (2) a site between the second and the third metatarsal heads and (3) a site where an extended line of inner periphery between the first and second and a perpendicular line of an inner ankle crosses.
